(19) United States
(12) Patent Application Publication
(10) Pub. No.: US 2004/0186085 A1
(43) Pub. Date: Sep. 23, 2004

(54) Composition containing a steroid and a glycol

(76) Inventor: Jean-Thierry Simonnet, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/484,428

(22) PCT Filed: Jul. 18, 2002
(86) PCT No.: PCT/FR02/02570

(30) Foreign Application Priority Data
Jul. 27, 2001 (FR) 01/10108

(51) Int. Cl. A61K 31/57

(52) U.S. Cl. 514/178

(57) ABSTRACT

The present invention relates to a composition comprising in a physiologically acceptable medium at least one steroid selected from DHEA and a metabolic or chemical derivative thereof, characterized in that the said composition further comprises dipropylene glycol.

Dipropylene glycol allows the steroid to be dissolved and its recrystallization to be avoided.

The invention also relates to cosmetic and dermatological uses of this composition, in particular for preventing or treating the signs of chronological or actinic skin ageing.

The present invention relates to a composition comprising in a physiologically acceptable medium at least one steroid selected from DHEA and a metabolic or chemical derivative thereof, characterized in that it further comprises dipropylene glycol.

DHEA, or dehydroepiandrosterone, is a natural steroid produced essentially by the adrenocortical glands. It is known for its capacity to promote the keratinization of the epidermis (JP-07 196 467), or else in the treatment of dry skin, by virtue of its capacity to increase the endogenous production and the secretion of sebum and to reinforce the skin's barrier effect (U.S. Pat. No. 4,496,556). Likewise described, in patent U.S. Pat. No. 5,843,932, has been the use of DHEA to remedy the atrophy of the dermis, by inhibiting the loss of collagen and of connective tissue. Finally, the use has been proposed of DHEA sulphate in order to treat various signs of ageing, such as wrinkles, loss of radiance of the skin, and skin slackening (EP-0 723 775).

Among the metabolites of DHEA, particular attention has been paid in recent years to 7α-hydroxy-DHEA. The reason for this is that it has been demonstrated that this metabolite, which does not possess the hormonal activity of DHEA, makes it possible to increase fibroblast proliferation and the viability of human keratinocytes, and has anti-free-radical effects (WO 98/40074). It has also been demonstrated in the rat (WO 00/28996) that 7α-hydroxy-DHEA increases the thickness of the dermis and the elastin content and collagen content of the skin. It has thus been suggested that this metabolite of DHEA be used to prevent and/or treat the harmful effects of UV radiation on the skin, to combat wrinkles and to increase the firmness and tone of the skin.

Understandable, therefore, is the interest that there may be in using DHEA and its derivatives in cosmetic or dermatological compositions.

However, these steroids do not dissolve readily in aqueous and aqueous-alcoholic media, thereby limiting their formulation in cosmetic or dermatological compositions. Thus they have a tendency to recrystallize. The formation of crystals of poorly controlled size, however, results in mediocre bioavailability of DHEA or its derivatives in the skin. The consequence is a greater or lesser loss of efficacy of the compositions comprising them, as a function of the degree of recrystallization, which runs counter to the desired objective. Moreover, this recrystallization may affect the overall stability of these compositions and also their appearance, which may turn the user away from them.

Solutions have thus been proposed by the Applicant to solubilize DHEA and its precursors and chemical and biological derivatives. Thus it has been suggested that DHEA be combined with 2-alkylalkanols or Guerbet alcohols (CA-2 343 426) or else with fat-soluble UV filters (FR 2 803 514).

However, for certain cosmetic applications of DHEA, these solutions are still not well suited. In particular, the use of fat-soluble UV filters in relatively large amount (greater than 10% by weight, for example) can lead to problems of intolerance and to a relatively greasy feel of the compositions comprising the DHEA, which is not always desirable from the cosmetic standpoint.

Moreover, the dissolving in Guerbet alcohols of the more polar derivatives of DHEA does not always lead to satisfactory results in emulsion. The reason for this is that these derivatives have a high capacity for what is termed Ostwald ripening, which leads to their rapid recrystallization in emulsion, by transfer of these derivatives from the fatty phase comprising them towards the aqueous phase of the emulsion.

There is therefore still a need to have compounds which allow DHEA and its derivatives to be dissolved, at the same time preventing their recrystallization in emulsion, which do not adversely affect the cosmetic properties, in particular the feel, of the emulsion obtained.

The Applicant has now found, surprisingly, that these steroids can be easily dissolved in certain glycols.

The present invention accordingly provides a composition comprising in a physiologically acceptable medium at least one steroid selected from DHEA and a metabolic or chemical derivative thereof, characterized in that the said composition further comprises dipropylene glycol.

The invention likewise relates to a method of dissolving at least one steroid selected from DHEA and/or a metabolic or chemical derivative thereof, which comprises the step consisting in mixing the said steroid into dipropylene glycol.

Mixing may be carried out cold, at ambient temperature or hot, for example at 75° C., generally with stirring.

DHEA has the formula (I):

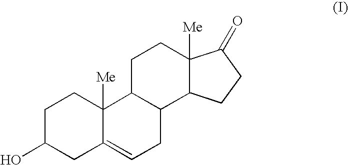

The DHEA which can be used according to the invention is, for example, available from Akzo Nobel.

By metabolic derivatives of DHEA are meant in particular 7α-OH-DHEA, 7β-OH-DHEA and 7-keto-DHEA, although this list is not limitative. 7α-OH-DHEA is preferred for use in the present invention.

By chemical derivative of DHEA is meant in particular the 3-alkyl esters of 7-oxo-DHEA and in particular 3β-acetoxy-7-oxo-DHEA, sold by Humanetics under the trade name 7-Keto®.

Other chemical derivatives of DHEA suitable for implementing the present invention are derivatives of formula (1):

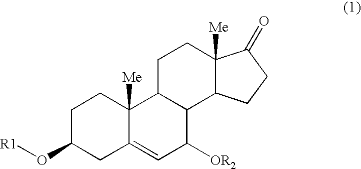

in which:

R₁ and R₂ are independently selected from:

a linear, branched or cyclic, saturated or unsaturated C₁-C₁₂ alkyl group which may optionally contain one or more heteroatoms and which is optionally substituted by one of more groups selected from —OR′ and/or —SR′ and/or —COOR′ and/or —NR′R′ and/or halogen and/or sulphate and/or phosphate and/or aryl and/or heterocycle, it being possible advantageously for the said heterocycle to be selected from an indole, a pyrimidine, a piperidine, a morpholine, a pyran, a furan, a piperazine and a pyridine;

an alkylcarbonyl group whose C₁-C₂₄ alkyl moiety is saturated or unsaturated, linear, branched or cyclic, and optionally substituted by one or more groups selected from —OR′ and/or —SR′ and/or —COOR′ and/or —NR′R′ and/or halogen and/or sulphate and/or phosphate and/or aryl and/or heterocycle, it being possible advantageously for the said heterocycle to be selected from an indole, a pyrimidine, a piperidine, a morpholine, a pyran, a furan, a piperazine and a pyridine; an arylcarbonyl group, preferably a phenylcarbonyl group, or an arylalkylcarbonyl group, preferably a benzylcarbonyl group, optionally substituted by one of more groups —OR′ and/or —SR′ and/or —COOR′ and/or —NR′R′ and/or halogen and/or aryl and/or heterocycle,

a group O═P(OH)OR′;

a group (O)₂SOR′;

a trialkylsilyl group (SiR′₃) in which the 3 groups R′ may be identical or different;

a carbonyloxyalkyl group (R′OCO);

a carbonylaminoalkyl group (R′NHCO); in which R′ is selected from a hydrogen atom, a linear, branched or cyclic, saturated or unsaturated C₁-C₁₂, preferably C₁-C₆, alkyl group which may, optionally contain one or more heteroatoms and is optionally functionalized by one or more groups —OR″, —COOR″, halogen, —NR″R″; or by an aryl group, preferably a phenyl group, optionally functionalized by one or more groups —OR″, —COOR″, halogen or —NR″R″, where R″ represents a hydrogen atom or a linear, branched or cyclic, saturated or unsaturated, preferably C₁-C₆, alkyl chain,

it being understood that in each of the groups —NR′R′ and —NR″R″ the substituents R′ and R″ respectively are identical or different.

Among the derivatives of formula (1) mention may be made in particular of the diesters of 7-OH-DHEA and more preferably 3-O-acetyl-7-benzoyloxydehydro-epiandrosterone, which is in particular available from Gattefosse under the trade name DHEA 3-acetoxy-7-benzoate.

The composition according to the present invention comprises, as solubilizer for the above steroids, dipropylene glycol.

This glycol may be used as the main solvent, in which case it allows the steroid to be dissolved in an aqueous medium, or as a secondary solvent. In this latter case, it may be added to a composition comprising fat-soluble UV filters or Guerbet alcohols as main solvents, in order to increase the solubility of the steroid in the aqueous phase when it is transferred to that phase from the oily phase containing the main solvent.

Dipropylene glycol therefore makes it possible both to solubilize the steroid, such as DHEA, and to stabilize it in respect of Ostwald ripening.

The concentration of steroid in the composition according to the invention is advantageously between 0.001% and 20% by weight, preferably between 0.01 and 10% by weight, more preferably between 0.1 and 5% by weight, relative to the total weight of the composition. Moreover, the amount by weight of glycol according to the invention represents advantageously from 1 to 40 times the amount by weight of steroid in the composition according to the invention; in other words, by way of indication, the glycol may represent from 0.01% to 50%, more preferably from 1 to 20%, of the total weight of the composition.

The composition according to the invention may be present in all of the pharmaceutical forms normally used for topical application to the skin, particularly in the form of an aqueous solution, a gel, an oil-in-water emulsion, an organic gel as described in application FR 2 794 998, a surfactant-free emulsion stabilized by polymer particles (as described in application EP-0 864 320) or by mineral particles (as described in applications WO 00/98301, WO 00/07548, WO 00/07549 and WO 00/07550), a microemulsion or a nanoemulsion. The content of these patents is incorporated here by reference.

This composition may be more or less fluid and may have the appearance of a white or coloured cream, an ointment, a milk, a lotion, a serum, a paste, a mousse or a gel. It may optionally be applied to the skin in aerosol form. It may be used as a care product and/or as a skin makeup product, or as a hair product, for example as a shampoo or conditioner.

Familiarly, the composition of the invention may likewise include the adjuvants customary in the fields of cosmetology and dermatology, such as hydrophilic or lipophilic gelling agents, hydrophilic or lipophilic actives, preservatives, antioxidants, solvents, perfumes, fillers, pigments, odour absorbers and colorants. The amounts of these various adjuvants are those conventionally used in the fields in question, and are for example from 0.01 to 20% of the total weight of the composition. These adjuvants, depending on their nature, may be introduced into the fatty phase or into the aqueous phase. These adjuvants and their concentrations must be such that they are not detrimental to the advantageous properties of the steroids according to the invention.

When the composition according to the invention is an emulsion the proportion of the fatty phase may range from 5 to 80% by weight and preferably from 5 to 50% by weight relative to the total weight of the composition. The fats, emulsifiers and coemulsifiers used in the composition in emulsion form are selected from those conventionally used in the field in question. The emulsifier and coemulsifier are preferably present in the composition in a proportion ranging from 0.3 to 30% by weight and preferably from 0.5 to 20% by weight relative to the total weight of the composition.

As fats which can be used in the invention it is possible to employ solubilizers based on 2-alkylalkanols and their esters, oils, and especially mineral oils (liquid paraffin), oils of plant origin (avocado oil, soya oil), oils of animal origin (lanolin), synthetic oils (perhydrosqualene), silicone oils (cyclomethicone) and fluoro oils (perfluoropolyethers). As fats it is also possible to use fatty alcohols such as cetyl alcohol, fatty acids, waxes and gums, and especially silicone gums.

As emulsifiers and coemulsifiers which can be used in the invention mention may be made, for example, of esters of fatty acid and polyethylene glycol, such as PEG-100 stearate, PEG-50 stearate and PEG-40 stearate; esters of fatty acid and polyols, such as glyceryl stearate, sorbitan tristerate and the ethoxylated sorbitan stearates available under the trade name Tween® 20 or Tween® 60, for example; alkyl ethers of saturated or unsaturated, linear or branched, C₁₀-C₂₀ fatty alcohols and polyethylene glycol, such as the PEG/stearyl alcohol ethers available under the trade name BRIJ 72 or BRIJ 721, for example; alkyl ethers of glycerol or of polyols; alkyl and/or polyalkyl ethers of glucose or of sucrose, such as the products sold under the names Crodesta F10, F20, F50, F70, F110 or F160, for example; and mixtures thereof.

As hydrophilic gelling agents mention may be made in particular of carboxyvinyl polymers (carbomers), acrylic copolymers, such as acrylate/alkyl acrylate copolymers, polyacrylamides, polysaccharides, natural gums and clays, and as lipophilic gelling agents mention may be made of modified clays such as Bentones, metal salts of fatty acids, and hydrophobic silica.

According to one preferred embodiment of the invention, the composition may comprise, in addition to dipropylene glycol, at least one other (poly)alkylene glycol and/or a (poly)alkylene glycol ester.

As examples of other alkylene glycols mention may be made of ethylene glycol, propylene glycol, butylene glycol, pentylene glycol and hexylene glycol.

As examples of other polyalkylene glycols mention may be made of polyethylene glycol containing from 2 to 20 units of ethylene glycol.

Finally, as (poly)alkylene glycol esters, mention may be made of the mono-, di- and triesters of C₁-C₆ acid and of alkylene glycol, such as propylene glycol acetate.

The composition according to the invention finds particular application in the prevention and/or treatment of the signs of chronological or actinic ageing.

The present invention accordingly likewise provides for the cosmetic use of the above-mentioned composition for preventing or treating the signs of chronological or actinic skin ageing.

It relates in particular to the cosmetic use of this composition for preventing or treating the loss of firmness of the skin and/or dull complexion and/or pore dilation and/or pigmentation disorders of the skin or hair.

The invention likewise provides for the cosmetic use of this composition for preventing or treating hyperseborrhoea and/or the imperfections associated with hyperseborrhoea and/or dandruff and/or hair loss.

The invention will now be illustrated by the following, non-limitative examples. In these examples the amounts are indicated as weight percentages unless indicated otherwise.

### EXAMPLES

### Example 1

### Solubilization of DHEA

The solubility of DHEA was evaluated at ambient temperature in different solvents. For this purpose the DHEA was dispersed in excess in the solvent in question, which had been brought to 60° C. beforehand. This temperature was maintained for one hour, with stirring using a magnetic bar. The suspension was subsequently returned to ambient temperature (25° C.). After 24 hours the suspension was centrifuged in order to remove the undissolved DHEA crystals. The supernatant was taken and checked for the absence of DHEA crystals by cross-polarized light microscopy. This supernatant was subsequently analysed by HPLC. The amount of DHEA detected is taken to correspond to its maximum solubility in the solvent in question.

The results are collated in the table below.

| | DHEA solubility |
|---|---|
| Solvent | (% by weight) |
| Glycerol | 0.15% |
| Sorbitol | 0.05% |
| PEG-400 | 3.67% |
| Propylene glycol | 5.88% |
| Dipropylene glycol | 10.48% |

It therefore emerges clearly from this table that dipropylene glycol is a much better solvent for DHEA than the solvents conventionally used in the cosmetic and pharmaceutical fields, such as glycerol and sorbitol, and even than other (poly)alkylene glycols such as polyethylene glycol (400 EO) and propylene glycol.

### Example 2

### Cosmetic Composition

| Phase A | |
|---|---|
| Glyceryl stearate | 2.5% |
| Polyethylene glycol stearate (8 EO) | 2.5% |
| Stearic acid | 1.0% |
| Preservative | 0.1% |
| Hexyldodecanol | 8.0% |
| Caprylic/capric triglycerides | 15% |
| DHEA | 1% |
| Phase B | |
| Triethanolamine | 0.25% |
| Preservative | 0.2% |
| Dipropylene glycol | 10% |
| Distilled water qs | 100% |
| Phase C | |
| Carbomer | 0.3% |
| Distilled water | 14.95% |
| Neutralizing agent | 0.25% |

The above composition may be prepared as follows. Phase A and phase B are heated separately to 75° C. Phase B is introduced into phase A with Moritz-type rotor-stator stirring. The temperature is held at 75° C. After 30 minutes of stirring, the composition is passed three times through a high-pressure homogenizer at between 200 bar and 900 bar. The suspension is subsequently brought to ambient temperature, after which phase C is dispersed using a deflocculator. A white emulsion of oil-in-water type is obtained which contains DHEA oleosomes.

This composition may be used to prevent or treat the signs of skin ageing such as wrinkles, fine lines and skin slackening.

### Example 3

### Cosmetic Composition

A lotion is prepared, conventionally for the person skilled in the art, which has the following composition:

| | |
|---|---|
| 3β-Acetoxy-7-oxo-DHEA | 1% |
| Dipropylene glycol | 25% |
| Propylene glycol | 20% |
| Glycerol | 30% |
| Distilled water | 100% |

This lotion can be used in particular to reduce pigmentary marks on the hands, neck and shoulders.

### Example 4

### Cosmetic Composition

An emulsion is prepared, in the same way as the composition of Example 2, which has the following composition:

| | |
|---|---|
| Phase A | |
| Diglyceryl distearate | 2.4% |
| Sorbitan stearate (4 EO) | 2.1% |
| Monosodium salt of N-stearoyl-L-glutamic acid | 0.7% |
| Cetostearyl alcohol | 1% |
| Vaseline | 2% |
| Stearyl heptanoate | 5% |
| Isocetyl stearate | 8% |
| Octyldodecanol | 8% |
| Capric/caprylic triglycerides | 5% |
| Cyclopentasiloxane | 5% |
| 7OH-DHEA | 0.8% |
| Phase B | |
| Dipropylene glycol | 25% |
| Preservatives | 1% |
| Distilled water | 25% |
| Phase C | |
| Carbomer | 0.2% |
| Triethanolamine | 0.1% |
| Water | |

This composition is highly suitable for very dry skin.

1. Composition comprising in a physiologically acceptable medium at least one steroid selected from DHEA and a metabolic or chemical derivative thereof, characterized in that the said composition further comprises dipropylene glycol.

2. Composition according to claim 1, characterized in that the said steroid is DHEA.

3. Composition according to claim 1, characterized in that the said metabolic derivative of DHEA is selected from 7αOH-DHEA, 7βOH-DHEA and 7-keto-DHEA.

4. Composition according to claim 1, characterized in that the said chemical derivative of DHEA is a 3-alkyl ester of 7-oxo-DHEA.

5. Composition according to claim 4, characterized in that the said chemical derivative of DHEA is 3β-acetoxy-7-oxo-DHEA.

6. Composition according to claim 1, characterized in that the said chemical derivative of DHEA is represented by the formula (1): 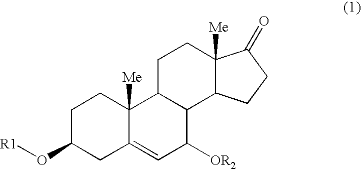
in which:
R₁ and R₂ are independently selected from:
a linear, branched or cyclic, saturated or unsaturated C₁-C₁₂ alkyl group which may optionally contain one or more heteroatoms and which is optionally substituted by one of more groups selected from —OR′ and/or —SR′ and/or —COOR′ and/or —NR′R′ and/or halogen and/or sulphate and/or phosphate and/or aryl and/or heterocycle, it being possible advantageously for the said heterocycle to be selected from an indole, a pyrimidine, a piperidine, a morpholine, a pyran, a furan, a piperazine and a pyridine;
an alkylcarbonyl group whose C₁-C₂₄ alkyl moiety is saturated or unsaturated, linear, branched or cyclic, and optionally substituted by one or more groups selected from —OR′ and/or —SR′ and/or —COOR′ and/or —NR′R′ and/or halogen and/or sulphate and/or phosphate and/or aryl and/or heterocycle, it being possible advantageously for the said heterocycle to be selected from an indole, a pyrimidine, a piperidine, a morpholine, a pyran, a furan, a piperazine and a pyridine;
an arylcarbonyl group, preferably a phenylcarbonyl group, or an arylalkylcarbonyl group, preferably a benzylcarbonyl group, optionally substituted by one of more groups —OR′ and/or —SR′ and/or —COOR′ and/or —NR′R′ and/or halogen and/or aryl and/or heterocycle;
a group O═P(OH)OR′;
a group (O)₂SOR′;
a trialkylsilyl group (SiR′₃) in which the 3 groups R′ may be identical or different;
a carbonyloxyalkyl group (R′OCO);
a carbonylaminoalkyl group (R′NHCO);
in which R′ is selected from a hydrogen atom, a linear, branched or cyclic, saturated or unsaturated C₁-C₁₂, preferably C₁-C₆, alkyl group which may optionally contain one or more heteroatoms and is optionally functionalized by one or more groups —OR″, —COOR″, halogen, —NR′R″; or by an aryl group, preferably a phenyl group, optionally functionalized by one or more groups —OR″, —COOR″, halogen or —NR″R″;
R″ represents a hydrogen atom or a linear, branched or cyclic, saturated or unsaturated, preferably C₁-C₆, alkyl chain,
it being understood that in each of the groups —NR″R″ and —NR′R′ the substituents R′ and R″ respectively are identical or different.

7. Composition according to claim 6, characterized in that the chemical derivative of DHEA is 3-O-acetyl-7-benzoyloxydehydroepiandrosterone.

8. Composition according to any one of claims 1 to 7, characterized in that it contains from 0.1 to 5% by weight of steroid relative to the total weight of the composition.

9. Composition according to any one of claims 1 to 8, characterized in that the amount by weight of glycol represents from 1 to 40 times the amount by weight of steroid in the composition.

10. Composition according to any one of claims 1 to 8, characterized in that the glycol represents from 1 to 20% of the total weight of the composition.

11. Composition according to any one of claims 1 to 10, characterized in that it further comprises at least one other (poly)alkylene glycol and/or a (poly)alkylene glycol ester.

12. Cosmetic use of the composition according to any one of claims 1 to 11 to prevent or treat the signs of chronological or actinic skin ageing.

13. Cosmetic use of the composition according to any one of claims 1 to 11 to prevent or treat loss of firmness of the skin and/or dull complexion and/or pore dilation and/or pigmentation disorders of the skin or hair.

14. Cosmetic use of the composition according to any one of claims 1 to 11 to prevent or treat hyperseborrhoea and/or imperfections linked to hyperseborrhoea and/or dandruff and/or hair loss.

15. Method of dissolving at least one steroid selected from DHEA and/or a metabolic or chemical derivative thereof, comprising the step consisting in mixing the said steroid into at least one glycol selected from hexylene glycol and dipropylene glycol.
